SEQUENCE LISTING (4 sequences)

SEQ ID NO: 1 - DNA, 27 bp, ARTIFICIAL SEQUENCE
  SYNTHETIC DNA
agaacatatgagaatcctcgtgatcaa

SEQ ID NO: 2 - DNA, 30 bp, ARTIFICIAL SEQUENCE
  SYNTHETIC DNA
aaaactgcagctagaggtactgcttctctg

SEQ ID NO: 3 - DNA, 711 bp, Arthrobacter aurescens
  CDS (1)..(711)
atgagaatcctcgtgatcaaccccaacagttccagcgcccttactgaatcggttgcggacgcagcacaacaagttgtcgcgaccggcaccataatttctgccatcaacccctccagaggacccgccgtcattgaaggcagctttgacgaagcactggccacgttccatctcattgaagaggtggagcgcgctgagcgggaaaacccgcccgacgcctacgtcatcgcatgtttcggggatccgggacttgacgcggtcaaggagctgactgacaggccagtggtaggagttgccgaagctgcaatccacatgtcttcattcgtcgcggccaccttctccattgtcagcatcctcccgagggtcaggaaacatctgcacgaactggtacggcaagcgggggcgacgaatcgcctcgcctccatcaagctcccaaatctgggggtgatggccttccatgaggacgaacatgccgcactggagacgctcaaacaagccgccaaggaggcggtccaggaggacggcgccgagtcgatagtgctcggatgcgccggcatggtggggtttgcgcgtcaactgagcgacgaactcggcgtccctgtcatcgaccccgtcgaggcagcttgccgcgtggccgagagtttggtcgctctgggctaccagaccagcaaagcgaactcgtatcaaaaaccgacagagaagcagtacctctag

SEQ ID NO: 4 - protein, 236 aa, Arthrobacter aurescens
MRILVINPNSSSALTESVADAAQQVVATGTIISAINPSRGPAVIEGSFDEALATFHLIEEVERAERENPPDAYVIACFGDPGLDAVKELTDRPVVGVAEAAIHMSSFVAATFSIVSILPRVRKHLHELVRQAGATNRLASIKLPNLGVMAFHEDEHAALETLKQAAKEAVQEDGAESIVLGCAGMVGFARQLSDELGVPVIDPVEAACRVAESLVALGYQTSKANSYQKPTEKQYL

(19) United States
(12) Patent Application Publication
(10) Pub. No.: US 2005/0244936 A1
(43) Pub. Date: Nov. 3, 2005

(54) Hydantoin-racemase

(75) Inventors: Josef Altenbuchner, Nufringen (DE); Ralf Mattes, Stuttgart (DE); Markus Pietzsch, Halle (DE); Christoph Syldatk, Stuttgart, DE (US); Anja Wiese, Eching (DE); Andreas Bommarius, Atlanta, GA (US); Wilhelm Tischer, Peissenberg (DE)

Correspondence Address: OBLON, SPIVAK, MCCLELLAND, MAIER & NEUSTADT, P.C., 1940 DUKE STREET, ALEXANDRIA, VA, 22314, US

(73) Assignees: DEGUSSA AG, Duesseldorf (DE); UNIVERSITAT STUTTGART, Stuttgart (DE)

(21) Appl. No.: 11/140,965

(22) Filed: Jun. 1, 2005

Related U.S. Application Data
(62) Division of application No. 10/088,093, filed on Sep. 30, 2002.

(30) Foreign Application Priority Data
Sep. 27, 1999 (EP) 99118956.4

(51) Int. Cl. C12P 13/04; C07H 21/04; C12N 9/90; C12N 15/74; C12N 1/21

(52) U.S. Cl. 435/106; 435/252.3; 435/471; 536/23.2; 536/24.3; 435/233

(57) ABSTRACT

The instant invention is directed to a rec-hydantoin-racemase from Arthrobacter aurescens DSM 3747. Furthermore, the gene encoding for the racemase and plasmids, vectors and microorganisms comprising this gene are to be protected. Use in a process for the production of amino carboxylic acids or derivatives thereof.

The instant invention is directed to a hydantoin-racemase from Arthrobacter aurescens (DSM 3747, hyuA).

The production of optically pure amino carboxylic acids is of growing interest in agrochemical, food and pharmaceutical industry. In particular, the enzymatic hydrolysis of hydantoins is an attractive method for the synthesis of D- and L-amino acids with regard to low-cost starting material and complete turnover of substrate.

Several hydantoin degrading micro-organisms have been isolated and the enzymatic conversion of 5′-monosubstituted hydantoins was studied in detail (Syldatk and Pietzsch, “Hydrolysis and formation of hydantoins” (1995), VCH Verlag, Weinheim, pp. 409-434; Ogawa et al., J. Mol. Catal. B: Enzym. 2 (1997), 163-176;Syldatk, C., May, O., Altenbuchner, J., Mattes, R. and Siemann, M. (1999) Microbiol. hydantoinases—industrial enzymes from the origin of life? Appl. Microbiol. Biotechnol. 51, 293-309). The asymmetric bio-conversion to either L- or D-amino acids consists of 3 steps:
(i) chemical and/or enzymatic racemization of 5′ substituted hydantoins,
(ii) ring opening hydrolysis achieved by a hydantoinase and
(iii) carbamoylase catalysed hydrolysis of the N-carbamoyl amino acid produced in the second step.

The chemical racemization of hydantoins proceeds via enolisation. The velocity depends on the electronic nature of the residue at the 5′-position (Ware, Chem. Rev. (1950), 46, 403-470) but usually, the racemization is a very slow process. For example, at room temperature and pH 8.5 only about 10% of L-IMH is racemized to D-IMH in 20 hour (Syldatk et al., “Biocatalytic production of amino acids and derivatives” (1992), Hanser publishers, New York, pp. 75-176). The rate of racemization is increased by a very basic pH (>10) and high temperature (>80° C.).

At physiological conditions a high rate of racemization is achieved by hydantoin-specific racemases. So far, hydantoin racemases have been purified and characterised from Arthrobacter (Syldatk et al., “Biocatalytic production of amino acids and derivatives” (1992), Hanser publishers, New York, pp. 75-176; Syldatk et al., “Hydrolysis and formation of hydantoins” (1995), VCH Verlag, Weinheim, pp. 409-434) and a Pseudomonas species (Watabe et al., J. Bacteriol. (1992a), 174, 3461-3466; Watabe et al., J. Bacteriol. (1992b), 174, 7989-7995). Only the latter is also characterised in terms of nucleotide sequence and genetic organisation.

It was, therefore, an object of this invention to provide another rec-hydantoin-racemase, which is able to racemize hydantoins under physiological conditions with an acceptable rate for their implementation in a process for the production of enantiomerically enriched amino carboxylic acids on industrial scale.

Providing the recombinantly derived hydantoin-racemase from Arthrobacter aurescens DSM 3747 (Seq. 4) is responsible for the dispense from above mentioned task. Especially, the racemase according to the invention can advantageously be incorporated in a large scale process for the production of enantiomerically enriched amino carboxylic acids. The feasibility of providing the racemase in a recombinant manner is the clue for acceptance of this process in view of economic efficiency.

Furthermore, a gene (Seq. 3) encoding for the racemase according to the invention is protected. The gene with relation to the framework of this invention is seen as a group of genes comprising all possible genes encoding for the protein in question according to the degeneration of the genetic code.

In another embodiment this invention encompasses plasmids, vectors and micro-organisms, which comprise the gene of instant invention. Within the framework of this invention all plasmids, vectors and micro-organisms which could advantageously be used to carry out the invention and are known to the skilled worker are incorporated herewith. Especially, those mentioned in Studier et al., Methods Enzymol. 1990, 185, 61-69 or those presented in brochures of Novagen, Promega, New England Biolabs, Clontech or Gibco BRL are deemed to be suitable. More applicable plasmids, vectors can be found in:
DNA cloning: a practical approach. Volume I-III, edited by D. M. Glover, IRL Press Ltd., Oxford, Washington D.C., 1985, 1987;
Denhardt, D. T. and Colasanti, J.: A surey of vectors for regulating expression of cloned DNA in E. coli. In: Rodriguez, R. L. and Denhardt, D. T (eds), Vectors, Butterworth, Stoneham, Mass., 1987, pp 179-204;
Gene expression technology. In: Goeddel, D. V. (eds), Methods in Enzymology, Volume 185, Academic Press, Inc., San Diego, 1990;
Sambrook, J., Fritsch, E. F. and Maniatis, T. 1989. Molecular cloning: a laboratory manual, 2^{nd} ed. Cold Spring Harbor Laboratory Press, Cold Spring Harbor, N.Y.

In addition, primers useful for the amplification of the gene of the invention in a PCR are protected similarly. Primers which are feasible are for example:

| | | |
|---|---|---|
| S1137 | 5′-AGAACATATGAGAATCCTCGTGATCAA-3′ | (Seq. 1) |
| S1138 | 5′-AAAACTGCAGCTAGAGGTACTGCTTCTCTG-3′ | (Seq. 2) |

Furthermore, all other primers which could serve to carry out this invention and which are known to the artisan are deemed to be useful in this sense. The finding of a suitable primer is done by comparison of known DNA-sequences or translation of amino acid sequences into the codon of the organism in question (e.g. for Streptomyceten: Wright et al., Gene 1992, 113, 55-65). Similarities in amino acid sequences of proteins of so called superfamilies are useful in this regard, too (Firestine et al., Chemistry & Biology 1996, 3, 779-783). Additional information can be found in Oligonucleotide synthesis: a practical approach, edited by M. J. Gait, IRL Press Ltd, Oxford Washington D.C., 1984; PCR Protocols: A guide to methods and applications, edited by M. A. Innis, D. H. Gelfound, J. J. Sninsky and T. J. White. Academic Press, Inc., San Diego, 1990. Those strategies are incorporated by reference herewith.

Another embodiment of this invention is the use of the racemase of the invention in a process for the production of amino carboxylic acids or derivatives thereof. Preferably, it is used according to the invention in a process for the production of enantiomerically enriched derivatives. Most preferably, the use is conducted in a covalent enzyme-membrane-reactor (DE19910691.6) or after non-covalent or covalent immobilisation to solid carriers (DE 197 033 14).

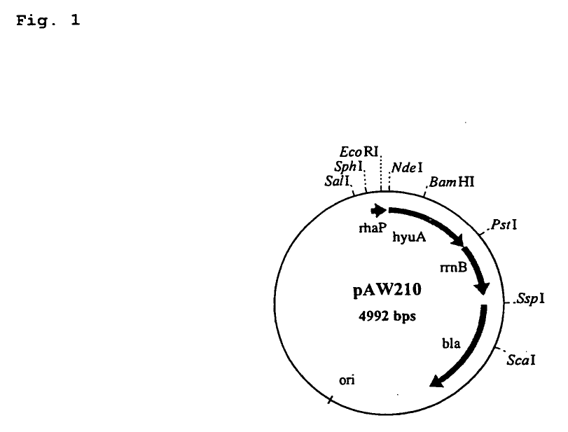

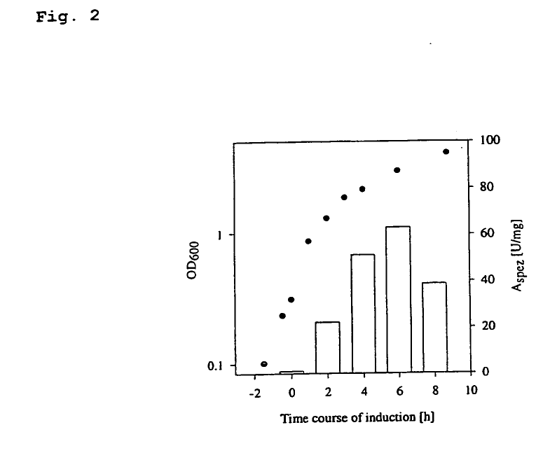

In order to prove the enzyme function, the gene was amplified by PCR from plasmid pAW16 using the primers S1137 and S1138 and placed under the control of a rhamnose promoter provided by the expression system pJOE2702. The resulting plasmid was designated pAW210 (FIG. 1). The E. coli cells harbouring pAW210 exhibited specific hydantoin racemase activities up to a maximum of 60 U/mg in crude cell extracts (FIG. 2). The racemase activity was determined in crude extracts by polarimetry using 3 mM L-BH as substrate (Teves et al., Presenius' J. Anal. Chem. 1999, 363, 738-743). An abundant protein of 31 kDa, representing approximately 10% of the total cellular protein, was detected by SDS-PAGE analysis in rhamnose induced cells and was mainly in the soluble fraction of the crude cell extracts.

The plasmid pAW210 in E. coli JM109 was used for purification of the racemase. A two step procedure consisting of ammonium sulfate fractionation and MonoQ anion exchange chromatography was accomplished as described down under. The racemase was purified 10-fold to homogeneity, with 35% overall recovery (Tab. 1).

TABLE 1
Purification of the racemase HyuA from E. Coli JM109 pAW210

| | | Protein- | Volumetric | Specific | Total | | |
|---|---|---|---|---|---|---|---|
| | Volume | con. | activity | activity | activity | Purification | Yield |
| Step | [ml] | [mg/ml] | [U/ml] | [U/mg] | [U] | [-fold] | [%] |
| Crude | 3 | 22.4 | 604 | 26.9 | 1812 | 1.0 | 100 |
| extract | | | | | | | |
| (NH₄)₂SO₄ | 2.5 | 7 | 317 | 45.2 | 792 | 1.7 | 44 |
| MonoQ^{a)} | 8.0 | 0.8 | 64 | 313.0 | 512 | 11.6 | 28 |

^{a)}Protein was purified on MonoQ in 4 separate runs using 4 mg for each run.

The specific activity of the purified enzyme was determined by standard enzyme assay with D-Benzylhydantoin as substrate at 313 U/mg. In potassium phosphate buffer, pH 7.0 with 25% glycerol, the purified enzyme could be stored for at least 6 months at −20° C. without noticeable loss of activity.

The matrix assisted laser desorption ionisation spectrum (MALDI) of the purified racemase gave a peak at a molecular mass of 25078.7. This is in good agreement with the calculated value of 25085 Da in contrast to the SDS-PAGE electrophoresis which gave a relative molecular mass of 31 kDa for the racemase monomer. On a calibrated column of superose 12 HR, the relative molecular mass of the native enzyme was estimated to be approximately 170 kDa±25. Due to the small subunit of 25 kDa and inaccuracy of the gel filtration method within this range the native enzyme is suggested to be either a hexamer, heptamer or octamer.

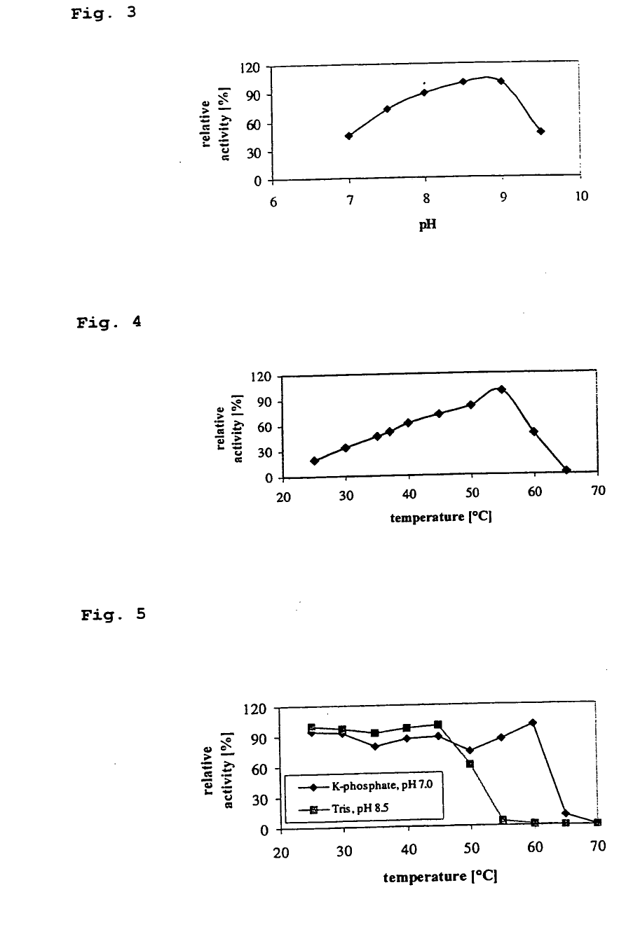

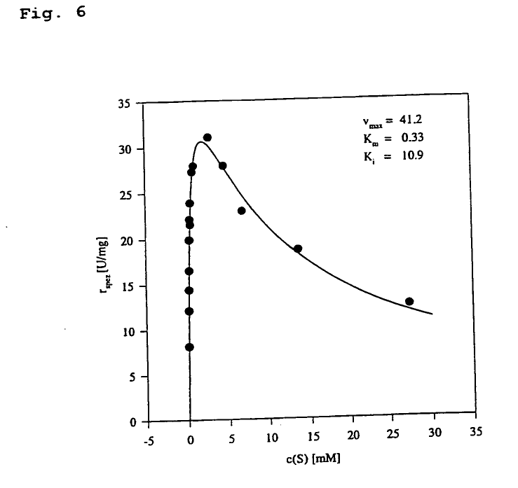

The effect of pH and temperature on the enzyme activity and stability are illustrated in FIG. 3-5. The pH optimum was determined between pH 8.0 and 9.0. Consequently, all standard assays were performed at pH 8.5. The optimum temperature for racemization of L-BH was around 55° C., however the stability of the enzyme under assay conditions (Tris, pH 8.5) was only maintained up to 45° C.

Racemization of the 5-substituted hydantoins BH, IMH and MTEH by HyuA was examined (Tab. 2).

TABLE 2
Substrate specificity of HyuA

| | Conc. | Relative Activity*) |
|---|---|---|
| Substrate | [mM] | [%] |
| L-MTEH | 0.9 | 7 |
| D-MTEH | 0.9 | 8 |
| L-BH | 0.9 | 100 |
| D-BH | 0.9 | 95 |
| L-IMH | 0.9 | 13 |
| D-IMH | 0.9 | 12 |

*)100% racemase activity corresponds to 313 μ/mg determined by standard assay

L- and D-BH gave the highest rates of activity, whereas the L- and D-isomer of MTEH were rather poorly racemised. indicating that aromatic hydantoins were preferred as substrates.

The K_{M} values of IMH and BH could not be determined due to the limited solubility of the substrates. Instead the initial velocities at different concentrations of L-MTEH were measured. The kinetic plot (FIG. 6) showed that the racemase is inhibited by the substrate L-MTEH. Even at low substrate concentrations (>5 mM) inhibition is observed.

The microorganism Arthrobacter aurescens used for the invention was desposited at Deutsche Sammlung für Mikroorganismen under the accession number DSM 3747.

### EXAMPLES

Bacterial strains, plasmids and growth conditions. E. coli JM109 (Yanisch-Perron et al., Gene (1985), 33, 103-109) was used for cloning, sequencing and expression the hyua gene from Arthrobacter aurescens DSM 3747 (Groβ et al., Biotech. Tech. (1987), 2, 85-90). E. coli strains were cultivated in 2xYT liquid broth or on 2xYT agar (Sambrook et al., Molecular Cloning: A Laboratory Manual (1989), Cold Spring Harbour Laboratory Press, New York). The media were supplemented with 100 μg/ml ampicillin to select plasmid carrying strains. The cultures were grown at 37° C., for hyuA expression the growth temperature was reduced to 30° C.

General protocols. All of the recombinant DNA techniques used were standard methods (Sambrook et al., Molecular Cloning: A Laboratory Manual (1989), Cold Spring Harbour Laboratory Press, New York). PCR reactions were performed with Taq DNA polymerase by following the recommendation by Roche Molecular Biochemicals. DNA sequencing was done from pUC-subclones with automated laser fluorescens DNA sequencer (Pharmacia LKB, Freiburg) by using AutoRead™ sequencing kit and M13 forward and reverse primer.

Expression of hyuA in E. coli. The racemase gene was amplified by PCR using the primers S1137 (5′-AGAACATATGAGAATCCTCGTGATCAA-3′) and S1138 (5′-AAAACTGCAGCTAGAGGTACTGCTTCTCTG-3′) and pAW16 as template (Wilms et al., J. Biotechnol. (1999), 68, 101-113). The fragment was inserted between the NdeI and PstI sites of the expression vector pJOE2702 (Volff et al., Mol. Microbiol. (1996), 21, 1037-1047) to create plasmid pAW210. Expression was induced by addition of 0.2% rhamnose to cultures at an optical density of 0.3 at 600 nm. After 6 h, cells corresponding to OD₆₀₀ of 10 were harvested, washed and resuspended in 1 ml desintegration buffer (0.07 M potassium phosphate, pH 7.0) and lysed by sonification (Ultrasonics sonicator, microtip, 2×30 s, duty cycle 50% pulsed). Clarified extracts were obtained by centrifugation at 14000 rpm for 10 min.

Enzyme assays. Racemization of L-BH was measured by ORD-polarimerty (Model 341, Perkin Elmer Bodenseewerk, Überlingen, Germany) at a wavelength of 295 nm in the standard assay for racemase enzyme activity. 3 MM L-BH was dissolved in 0.1 M Tris, pH 3 at 45° C. in an ultrasonic waterbath, cooled to room temperature and the pH adjusted to pH 8.5 with 3 M NaOH. To 1 ml substrate solution 0.1 ml enzyme, diluted in 0.1 M Tris, pH 8.5, was added and the change in optical rotation determined at 37° C. by polarimetry (Teves et al., Fresenius' J. Anal. Chem. 1999, 363, 738-743). The racemization of MTEH and IMH by. HyuA was determined at substrate concentrations of 0.9 mM and recorded by ORD at 253 nm and 334 nm. The specific activities were calculated from initial reaction rates which were determined according to Teves et al. (1999). For determination of enzyme activity by HPLC, 1 mM L-IMH was dissolved as described above. The mixture containing 900 μl enzyme solution was incubated 5 min at 37° C. The reaction was stopped by addition of 400 μl 14% trichloroacetic acid and centrifugation in an Eppendorf centrifuge at full speed. 100 μl of the sample were diluted with 0.9 ml 0.1 M TrisHCl, pH 8.5, and D-IMH and L-IMH in the supernatant were separated by HPLC (Thermoseparation Products, Darmstadt, Germany) by injection of 20 μl sample into a Chiralpak WH-column (0.46×25 cm; Daicel Chemicals Industris LTD, Griesheim, Germany). The column was equilibrated with 0.25 mM CuSO₄, pH 5.5. The flow rate was 1 ml/min at 50° C. and IMH detected at 254 nm. The chemical racemization of the substrate was taken into account. Racemization of 1 μm substrate per minute was defined as one unit enzyme.

Purification of recombinant hyua. For the preparation of crude extract, cells from 300 ml culture of rhamnose induced E. coli JM109 pAW210 were resuspended in 3 ml desintegration buffer and disrupted 3 times by french press (Amico, SLM Instruments Inc, Illinois, USA) at a pressure of 600 bar. Solid (NH₄)₂SO₄ was gradually added to the cell-free extract to a concentration of 1.5 M and stired 2 h at 4° C. The precipitate formed was removed by centrifugation (Sorvall) and discarded. Another 0.7 M (NH₄)₂SO₄ was added to the supernatant. The second precipitate obtained by centrifugation was resuspended in buffer A (10 mM potassium phosphate, pH 6.5) and applied to a MonoQ® HR 5/5 column equilibrated in buffer A and eluted with a linear gradient of 0 to 1.0 M NaCl in buffer A. HyuA was eluted at a concentration of 0.37 M NaCl. Peak fractions were pooled and dialyzed against desintegration buffer, glycerol was added to a final concentration of 25% and stored at −20° C.

Protein characterisation. Sodium dodecyl sulfate polyacrylamide gel electrophoresis (SDS-PAGE) was done according to the method of Laemmli (Laemmli, Nature (1970), 227, 680-685). Protein concentrations were determined by the method of Bradford (Bradford, Anal. Biochem. (1976), 72, 248-254) using the Biorad protein assay dye reagent concentrate. Standard curves were generated with bovine serum albumin. The Mᵣ of native protein was determined by gel filtration using superose 12HR column as described previously (Wilms et al., J. Biotechnol. (1999), 68, 101-113), the column was equilibrated and eluted with buffer consisting of 0.1 M potassium phosphate and 0.1 M NaCl, pH 7. The pH profile of the purified racemase was measured between the pH range 7.0 to 9.5 in Tris buffer. The substrate was dissolved in 0.1 M Tris, pH 3 at 45° C. using an ultrasonic waterbath. After cooling to room temperature, the pH was adjusted to the desired pH with sodium hydroxide and enzyme activity was determined using the standard assay. The reaction temperature optimum of purified racemase was determined using temperatures between 25 and 65° C. in the standard assay. The stability of the enzyme was measured after preincubation at temperatures between 25 and 70° C. for 15 minutes in the presence of desintegration buffer and 0.1 M Tris buffer, pH 8.5, respectively. The increased chemical racemization at high pH and temperatures, respectively, was considered. The effect of EDTA, DTT, HgCl₂ and iodoacetamid on HyuA was tested by incubation of respective substance (10 mM) and purified enzyme (12 μg) in desintegration buffer (final volume 20 μl) at 30° C. After 1 h specific activities were determined by the standard enzyme assay.

1-8. (canceled)

9. An isolated polynucleotide encoding a rec-Hydantoin-racemase from Arthrobacter auresens DSM 3747.

10. A vector comprising the isolated polynucleotide of claim 9.

11. A microorganism comprising the isolated polynucleotide of claim 9.

12. A primer for the isolated polynucleotide of claim 9.

13. A method of producing amino carboxylic acids or a derivative thereof, comprising culturing the microorganism of claim 11 and collecting the amino carboxylic acids or a derivative thereof.

14. The method of claim 13, wherein enatiomerically enriched amino carboxylic acids or derivatives thereof are produced.

15. The method of claim 13, wherein the culturing is conducted in an enzyme-membrane-reactor.

16. The method of claim 14, wherein the culturing is conducted in an enzyme-membrane-reactor.
